(19) United States
(12) Patent Application Publication
(10) Pub. No.: US 2026/0011782 A1
(43) Pub. Date: Jan. 8, 2026

(54) COMPOUND, ELECTROLYTE FOR RECHARGEABLE LITHIUM BATTERY INCLUDING THE SAME, AND RECHARGEABLE LITHIUM BATTERY INCLUDING THE SAME

(71) Applicant: SAMSUNG SDI CO., LTD., Yongin-si (KR)

(72) Inventors: SUNDAE KIM, Yongin-si (KR); Euisoo JEONG, Yongin-si (KR); JIN-HYEOK LIM, Yongin-si (KR); JINAH SEO, Yongin-si (KR); Erang CHO, Yongin-si (KR); Bomee KWON, Yongin-si (KR); Sujeong KOH, Yongin-si (KR); SANGHOON KIM, Yongin-si (KR)

(21) Appl. No.: 19/093,077

(22) Filed: Mar. 27, 2025

(30) Foreign Application Priority Data
Jul. 2, 2024 (KR) 10-2024-0086740

(51) Int. Cl. H01M 10/0567; C07D 285/10; H01M 4/525; H01M 10/0525; H01M 10/0568; H01M 10/0569

(52) U.S. Cl. CPC H01M 10/0567; C07D 285/10; H01M 4/525; H01M 10/0525; H01M 10/0568; H01M 10/0569; H01M 2300/004

(57) ABSTRACT

A compound, an electrolyte including the compound, and a rechargeable lithium battery including the electrolyte are provided. The electrolyte may include a non-aqueous organic solvent, a lithium salt, and an additive, and the additive may include the compound that is represented by Formula 1.

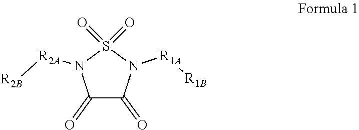

### CROSS-REFERENCE TO RELATED APPLICATION

The present application claims priority to and the benefit of Korean Patent Application No. 10-2024-0086740, filed on Jul. 2, 2024, in the Korean Intellectual Property Office, the entire disclosure of which is incorporated herein by reference.

### BACKGROUND

### 1. Field

One or more embodiments of the present disclosure herein relate to a compound, an electrolyte for a rechargeable lithium battery including the compound, and a rechargeable lithium battery including the electrolyte.

### 2. Description of the Related Art

Recently, with the rapid spread and popularization of electronic devices that use batteries (such as mobile phones and/or laptop computers) and/or of electric vehicles, the demand for batteries, for example rechargeable batteries, with relatively high energy density and high capacity is rapidly increasing. Accordingly, there is active research and development aimed at improving or enhancing the performance of such rechargeable batteries, e.g., rechargeable lithium batteries.

A rechargeable lithium battery is a battery including a positive electrode and a negative electrode, both containing active materials capable of intercalation and deintercalation of lithium ions, along with an electrolyte. Electrical energy is produced (generated) through the oxidation and reduction reactions as lithium ions are intercalated into and deintercalated from the positive electrode and negative electrode (e.g., intercalated into the positive electrode and/or deintercalated from the negative electrode during the discharge process).

The electrolyte of the rechargeable lithium batteries uses or includes a lithium salt dissolved in a non-aqueous organic solvent. The rechargeable lithium battery's properties are achieved through complex reactions between the positive electrode and electrolyte, as well as between the negative electrode and electrolyte. Therefore, the use of an appropriate or suitable electrolyte is an important variable that may improve the performance of rechargeable lithium batteries.

### SUMMARY

One or more aspects of embodiments of the present disclosure are directed toward an additive that enhances the performance of rechargeable batteries at elevated or high temperatures (i.e., has excellent or suitable improving effects on the performance of rechargeable lithium batteries at high temperatures).

One or more aspects of embodiments of the present disclosure are directed toward a rechargeable lithium battery having excellent or suitable performance at high temperatures by including the additive.

Additional aspects will be set forth in part in the description which follows and, in part, will be apparent from the description, or may be learned by practice of the presented embodiments of the disclosure.

According to one or more embodiments of the present disclosure, an electrolyte for a rechargeable lithium battery may include a non-aqueous organic solvent, a lithium salt, and an additive, wherein the additive is a compound represented by Formula 1.

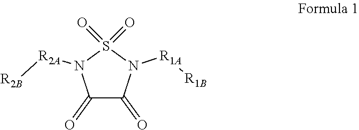

In Formula 1,
R_{1A} and R_{2A} may each independently be a substituted or unsubstituted C1 to C10 alkylene group, a substituted or unsubstituted C2 to C10 alkenylene group, or a substituted or unsubstituted C2 to C10 alkynylene group,

R_{1A} and R_{2A} may each independently be hydrogen, a substituted or unsubstituted C1 to C10 alkyl group, a substituted or unsubstituted C2 to C10 alkenyl group, or a substituted or unsubstituted C2 to C10 alkynyl group, and at least one selected from among R_{1B} and R_{2B} may include an alkenyl group.

### BRIEF DESCRIPTION OF DRAWINGS

The accompanying drawings are included to provide a further understanding of the present disclosure, and are incorporated in and constitute a part of this disclosure. The drawings illustrate example embodiments of the present disclosure and, together with the description, serve to explain principles of the present disclosure. The above and other aspects, features, and advantages of certain embodiments of the present disclosure will be more apparent from the following description taken in conjunction with the accompanying drawings.

FIG. 1 is a simplified conceptual diagram showing a rechargeable lithium battery according to one or more embodiments of the present disclosure.

FIGS. 2-5 are each a simplified schematic diagram showing a rechargeable lithium battery according to one or more embodiments of the present disclosure, where FIG. 2 illustrates a cylindrical battery, FIG. 3 illustrates a prismatic battery, and FIGS. 4 and 5 each illustrate a pouch-type (kind) battery.

FIG. 6 illustrates proton nuclear magnetic resonance (¹H-NMR) spectroscopy results on a compound according to Synthetic Example 1 of the present disclosure.

### DETAILED DESCRIPTION

In order to sufficiently understand the configurations and effects of the present disclosure, one or more embodiments of the present disclosure will be described with reference to the accompanying drawings. It should be noted, however, that the present disclosure is not limited to the following example embodiments, and may be implemented in one or more other suitable forms. Rather, the example embodiments are provided only to illustrate the present disclosure and assist those skilled in the art to fully know the scope of the disclosure.

In the present disclosure, it will be understood that, if (e.g., when) an element is referred to as being on another element, the element may be directly on the other element or intervening elements may be present therebetween. In contrast, if (e.g., when) an element is referred to as being “directly on” another element, there are no intervening elements present. In the drawings, thicknesses of some components may be exaggerated for effectively explaining the technical contents. Like reference numerals refer to like elements throughout the present disclosure, and duplicative descriptions thereof may not be provided for conciseness.

Unless otherwise specially noted in the disclosure, the singular forms “a,” “an,” and “the” are intended to include the plural forms as well, unless the context clearly indicates otherwise. Further, the utilization of “may” when describing embodiments of the present disclosure refers to “one or more embodiments of the present disclosure”. In addition, unless otherwise specially noted, the phrase “A or B” or “A and/or B” or “A/B” may indicate “A but not B,” “B but not A,” and “A and B.” The terms “comprise(s)/include(s)” and/or “comprising/including” and/or “have(has)/having used in the present disclosure do not exclude the presence or addition of one or more other components.

As used herein, the term “combination thereof” may refer to a mixture, a stack, a composite, a copolymer, an alloy, a blend, or a reaction product of the constituents.

Unless otherwise especially defined in the disclosure, a particle diameter

may be an average particle diameter. In addition, a particle diameter indicates an average particle diameter (D₅₀) where a cumulative volume is about 50 volume % in a particle size distribution. The average particle diameter (D₅₀) may be measured by a method widely suitable to those skilled in the art, for example, by a particle size analyzer, for example, HORIBA, LA-950 laser particle size analyzer, a transmission electron microscope (TEM), or a scanning electron microscope (SEM). In one or more embodiments, a dynamic light-scattering measurement device is used to perform a data analysis, the number of particles is counted for each particle size range, and then from the collected data, an average particle diameter (D₅₀) value may be obtained through a calculation. In some embodiments, a laser scattering method may be utilized to measure an average particle diameter (D₅₀). In the laser scattering method, target particles are distributed in a dispersion solvent, introduced into a laser scattering particle measurement device (e.g., MT3000 commercially available from Microtrac, Inc), irradiated with ultrasonic waves of 28 kHz at a power of 60 W, and then an average particle diameter (D₅₀) is calculated in the 50% standard of particle diameter distribution in the measurement device. D50 refers to the average diameter (or size) of particles whose cumulative volume corresponds to 50 vol % in the particle size distribution (e.g., cumulative distribution), and refers to the value of the particle size corresponding to 50% from the smallest particle when the total number of particles is 100% in the distribution curve accumulated in the order of the smallest particle size to the largest particle size. In the present disclosure, when particles are spherical, “diameter” indicates an average particle diameter, and when the particles are non-spherical, the “diameter” indicates a major axis length.

In the present disclosure, unless otherwise separately defined, the term “substituted” may refer to that at least one hydrogen of a substituent or a compound is substituted by deuterium, a halogen, a hydroxyl group, an amino group, a C1 to C30amine group, a nitro group, a C1 to C40 silyl group, a C1 to C30 alkyl group, a C1 to

C10 alkylsilyl group, a C6 to C30 arylsilyl group, a C3 to C30 cycloalkyl group, a C3 to C30 heterocycloalkyl group, a C6 to C30 aryl group, a C2 to C30 heteroaryl group, C1to C20 alkoxy group, a C1 to C10 fluoroalkyl group, a cyano group, and/or a (e.g., any suitable) combination thereof.

In more detail, the term “substituted” may refer to that at least one hydrogen of a substituent or a compound is substituted by deuterium, a halogen, a C1 to C30alkyl group, a C1 to C10 alkylsilyl group, a C6 to C30 arylsilyl group, a C3 to C30cycloalkyl group, a C3 to C30 heterocycloalkyl group, a C6 to C30 aryl group, a C2 to C30 heteroaryl group, a C1 to C10 fluoroalkyl group, or a cyano group. For example, the term “substituted” may refer to that at least one hydrogen of a substituent or a compound is substituted by deuterium, a halogen, a C1 to C20 alkyl group, a C6 to C30 aryl group, a C1 to C10 fluoroalkyl group, or a cyano group. In one or more embodiments, the term “substituted” may refer to that at least one hydrogen of a substituent or a compound is substituted by deuterium, a halogen, a C1 to C5 alkyl group, a C6 to C18 aryl group, a C1 to C5 fluoroalkyl group, or a cyano group. For example, the term “substituted” may refer to that at least one hydrogen of a substituent or a compound is substituted by deuterium, a cyano group, a halogen, a methyl group, an ethyl group, a propyl group, a butyl group, a phenyl group, a biphenyl group, a terphenyl group, a trifluoromethyl group, or a naphthyl group.

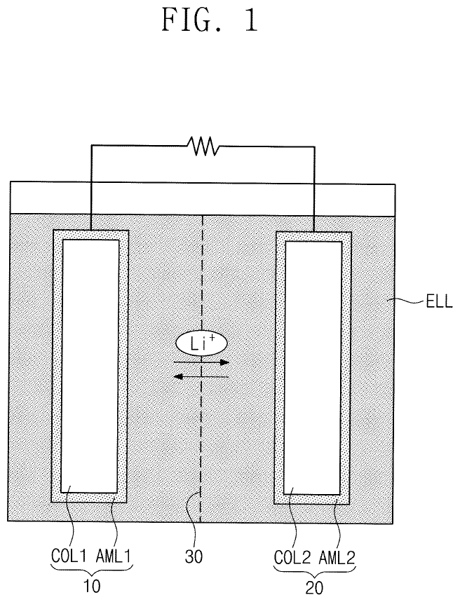

FIG. 1 illustrates a simplified conceptual diagram showing a rechargeable lithium battery according to one or more embodiments of the present disclosure. Referring to FIG. 1, a rechargeable lithium battery may include a positive electrode 10, a negative electrode 20, a separator 30, and an electrolyte ELL.

The positive electrode 10 and the negative electrode 20 may be spaced and/or apart (e.g., spaced apart or separated) from each other across the separator 30. The separator 30 may be arranged between the positive electrode 10 and the negative electrode 20. The positive electrode 10, the negative electrode 20, and the separator 30 may be in contact with the electrolyte ELL. For example, the positive electrode 10, the negative electrode 20, and the separator 30 may be impregnated in and/or with the electrolyte ELL.

The electrolyte ELL may be a medium through which lithium ions are migrated and transferred between the positive electrode 10 and the negative electrode 20. In the electrolyte ELL, the lithium ions may move through the separator 30 toward one selected from among the positive electrode 10 and the negative electrode 20.

### Positive Electrode 10

The positive electrode 10 for a rechargeable lithium battery may include a

current collector COL1 and a positive electrode active material layer AML1 formed on the current collector COL1. The positive electrode active material layer AML1 may include a positive electrode active material (e.g., in a form of particles) and may further include a binder and/or a conductive material (e.g., an electron conductor). An amount of the positive electrode active material may be in a range of

about 90 wt % to about 99.5 wt % relative to (i.e., based on) 100 wt % of a total weight of the positive electrode active material layer AML1. An amount of each of the binder and the conductive material may be in a range of about 0.5 wt % to about 5 wt % relative to (i.e., based on) 100 wt % of the total weight of the positive electrode active material layer AML1.

The binder may serve to improve attachment of positive electrode active material particles to each other and also to improve attachment of the positive electrode active material to the current collector COL1. The binder may include, for example, one or more selected from among polyvinyl alcohol, carboxymethyl cellulose, hydroxypropyl cellulose, diacetyl cellulose, polyvinylchloride, carboxylated polyvinylchloride, polyvinylfluoride, an ethylene oxide-containing polymer, polyvinylpyrrolidone, polyurethane, polytetrafluoroethylene, polyvinylidene fluoride, polyethylene, polypropylene, a styrene-butadiene rubber, a (meth)acrylated styrene-butadiene rubber, an epoxy resin, a (meth)acrylic resin, a polyester resin, and nylon, but embodiments of the present disclosure are not limited thereto.

The conductive material (e.g., an electrically or electron conductive material or conductor) may be used to provide an electrode with conductivity, and any suitable conductive material that does not cause a chemical change in a battery may be used as the conductive material. The conductive material may include, for example, a carbon-based material such as natural graphite, artificial graphite, carbon black, acetylene black, Ketjen black, carbon fiber, carbon nano-fiber, and/or carbon nano-tube; a metal powder or metal fiber containing one or more of (e.g., selected from among) copper, nickel, aluminum, and silver; a conductive polymer such as a polyphenylene derivative; and/or a (e.g., any suitable) mixture thereof.

In one or more embodiments, aluminum (Al) may be used as the current collector COL1, but embodiments of the disclosure are not limited thereto.

### Positive Electrode Active Material

The positive electrode active material in the positive electrode active material layer AML1 may include a compound (e.g., lithiated intercalation compound) that may reversibly intercalate and deintercalate lithium. For example, in one or more embodiments, the positive electrode active material may include at least one kind of composite oxide including lithium and a metal that is selected from among cobalt, manganese, nickel, and/or a (e.g., any suitable) combination thereof.

The composite oxide may include lithium transition metal composite oxides, for example, lithium-nickel-based oxides, lithium-cobalt-based oxides, lithium-manganese-based oxides, lithium-iron-phosphate-based compounds, cobalt-free nickel-manganese-based oxides, and/or a (e.g., any suitable) combination thereof.

For example, in one or more embodiments, the positive electrode active material may include a compound expressed by one selected from among chemical formulae: LiₐA_{1-b}X_{b}O_{2-c}D_{c} (where 0.90≤a≤1.8, 0≤b≤0.5, and 0≤c≤0.05); LiₐMn_{2-b}X_{b}O_{4-c}D_{c} (where 0.90≤a≤1.8, 0b≤0.5, and 0≤c≤0.05); LiₐNi_{1-b-c}Co_{b}X_{c}O_{2-α}D_{α} (where 0.90≤a≤1.8, 0≤b≤0.5, 0≤c≤0.5, and 0<α<2); LiₐNi_{1-b-c}Mn_{b}X_{c}O_{2-α}D_{α} (where 0.90≤a≤1.8, 0≤b≤0.5, 0≤c≤0.5, and 0<α<2); LiₐNi_{b}Co_{c}L¹_{d}GₑO₂ (where 0.90≤a≤1.8, 0≤b≤0.9, 0≤c≤0.5, 0≤d≤0.5, and 0≤e≤0.1); Li^{a}NiG_{b}O₂ (where 0.90≤a≤1.8 and 0.001≤b≤0.1); LiₐCoG_{b}O₂(where 0.90≤a≤1.8 and 0.001≤b≤0.1); LiₐMn_{1-b}G_{b}O₂ (where 0.90≤a≤1.8 and 0.001≤b≤0.1); LiₐMn₂G_{b}O₄ (where 0.90≤a≤1.8 and 0.001≤b≤0.1); LiₐMn₁₋₉G_{g}PO₄ (where 0.90≤a≤1.8 and 0≤g≤0.5); Li(_{3-f})Fe₂(PO₄)₃ (where 0≤f≤2); LiₐFₑPO₄ (where 0.90≤a≤1.8).

In the foregoing chemical formulae, A may be nickel (Ni), cobalt (Co), manganese (Mn), or a (e.g., any suitable) combination thereof, X may be Al, Ni, Co, Mn, chromium (Cr), iron (Fe), magnesium (Mg), strontium (Sr), vanadium (V), a rare-earth element, or a (e.g., any suitable) combination thereof, D may be oxygen (O), fluorine (F), sulfur(S), phosphorous (P), or a (e.g., any suitable) combination thereof, G may be Al, Cr, Mn, Fe, Mg, lanthanum (La), cerium (Ce), Sr, V, or a (e.g., any suitable) combination thereof, and L1 may be Mn, Al, or a (e.g., any suitable) combination thereof.

For example, in one or more embodiments, the positive electrode active material may be a high-nickel-based positive electrode active material having a nickel amount of equal to or greater than about 80 mol %, equal to or greater than about 85 mol %, equal to or greater than about 90 mol %, equal to or greater than about 91mol %, or equal to or greater than about 94 mol % and equal to or less than about 99 mol % relative to (i.e., based on) 100 mol % of a total metal excluding lithium in the lithium transition metal composite oxide. The high-nickel-based positive electrode active material may achieve high capacity and thus may be applied to a high-capacity and high-density rechargeable lithium battery.

### Negative Electrode 20

The negative electrode 20 for a rechargeable lithium battery may include a current collector COL2 and a negative electrode active material layer AML2 positioned on the current collector COL2. The negative electrode active material layer AML2 may include a negative electrode active material (e.g., in a form of particles) and may further include a binder and/or a conductive material (e.g., an electron conductor).

For example, in one or more embodiments, the negative electrode active material layer AML2 may include a negative electrode active material of about 90 wt % to about 99 wt %, a binder of about 0.5 wt % to about 5 wt %, and a conductive material of about 0 wt % to about 5 wt %, based on 100 wt % of a total weight of the negative electrode active material layer.

The binder may serve to improve attachment of negative electrode active material particles to each other and also to improve attachment of the negative electrode active material to the current collector COL2. The binder may include a non-aqueous (e.g., water-insoluble) binder, an aqueous (e.g., water-soluble) binder, a dry binder, and/or a (e.g., any suitable) combination thereof.

The non-aqueous binder may include polyvinyl chloride, carboxylated polyvinyl chloride, polyvinyl fluoride, an ethylene propylene copolymer, polystyrene, polyurethane, polytetrafluoroethylene, polyvinylidene fluoride, polyethylene, polypropylene, polyamide imide, polyimide, and/or a (e.g., any suitable) combination thereof.

The aqueous binder may include a styrene-butadiene rubber, a (meth)acrylated styrene-butadiene rubber, a (meth)acrylonitrile-butadiene rubber, a (meth)acrylic rubber, a butyl rubber, a fluoro elastomer, polyethylene oxide, polyvinyl pyrrolidone, polyepichlorohydrin, polyphosphazene, poly (meth)acrylonitrile, an ethylene propylene diene copolymer, polyvinyl pyridine, chlorosulfonated polyethylene, latex, a polyester resin, a (meth)acrylic resin, a phenolic resin, an epoxy resin, polyvinyl alcohol, and/or a (e.g., any suitable) combination thereof.

When an aqueous binder is used as the binder of the negative electrode, a

cellulose-based compound capable of providing viscosity may further be included. The cellulose-based compound may include one or more selected from among carboxymethyl cellulose, hydroxypropylmethyl cellulose, methyl cellulose, and alkali metal salts thereof. The alkali metal may include Na, K, or Li.

The dry binder may include a fibrillizable polymer material, for example, polytetrafluoroethylene, polyvinylidene fluoride, a polyvinylidene fluoride-hexafluoropropylene copolymer, polyethylene oxide, and/or a (e.g., any suitable) combination thereof.

The conductive material (e.g., electrically or electron conductive material or conductor) may be used to provide an electrode with conductivity, and any suitable conductive material that does not cause a chemical change in a battery may be used as the conductive material. For example, in one or more embodiments, the conductive material may include a carbon-based material such as natural graphite, artificial graphite, carbon black, acetylene black, Ketjen black, carbon fiber, carbon nano-fiber, and/or carbon nano-tube; a metal powder or metal fiber including one or more selected from among copper, nickel, aluminum, and silver; a conductive polymer such as a polyphenylene derivative; and/or a (e.g., any suitable) mixture thereof.

The current collector COL2 may include a copper foil, a nickel foil, a stainless-steel foil, a titanium foil, a nickel foam, a copper foam, a polymer substrate coated with a conductive metal, and/or a (e.g., any suitable) combination thereof.

### Negative Electrode Active Material

The negative electrode active material in the negative electrode active material layer AML2 may include a material that may reversibly intercalate and deintercalate lithium ions, lithium metal, a lithium metal alloy, a material that may dope and de-dope lithium, or a transition metal oxide.

The material that may reversibly intercalate and deintercalate lithium ions may include a carbon-based negative electrode active material, for example, crystalline carbon, amorphous carbon, and/or a (e.g., any suitable) combination thereof. For example, the crystalline carbon may include graphite such as non-shaped (e.g., irregularly shaped), sheet-shaped, flake-shaped, sphere-shaped, or fiber-shaped natural graphite and/or artificial graphite, and the amorphous carbon may include soft carbon, hard carbon, mesophase pitch carbon, and/or calcined coke.

The lithium metal alloy may include an alloy of lithium and a metal that is selected from among sodium (Na), potassium (K), rubidium (Rb), cesium (Cs), francium (Fr), beryllium (Be), magnesium (Mg), calcium (Ca), strontium (Sr), silicon (Si), antimony (Sb), lead (Pb), indium (In), zinc (Zn), barium (Ba), radium (Ra), germanium (Ge), aluminum (AI), and tin (Sn).

The material that may dope and de-dope lithium may include a Si-based negative electrode active material or a Sn-based negative electrode active material. The Si-based negative electrode active material may include silicon, a silicon-carbon composite, SiOₓ (where 0<x≤2), a Si—Q alloy (where Q is an alkali metal, an alkaline earth metal, a Group 13 element, a Group 14 element (except for Si), a Group 15element, a Group 16 element, a transition metal, a rare-earth element, or a (e.g., any suitable) combination thereof), and/or a (e.g., any suitable) combination thereof. The Sn-based negative electrode active material may include Sn, SnOₖ (0<k≤2) (e.g., SnO₂), a Sn-based alloy, or a (e.g., any suitable) combination thereof. The silicon-carbon composite may be a composite of silicon and amorphous carbon (e.g., in a form of particles). According to one or more embodiments, the silicon-carbon composite may have a structure in which the amorphous carbon is coated on a surface of the silicon particle. For example, in one or more embodiments, the silicon-carbon composite may include a secondary particle (core) in which primary silicon particles are assembled, and an amorphous carbon coating layer (shell) on (e.g., positioned on) a surface of the secondary particle. The amorphous carbon may also be positioned between the primary silicon particles, and for example, the primary silicon particles may be coated with the amorphous carbon. The secondary particles may be present dispersed in an amorphous carbon matrix.

In one or more embodiments, the silicon-carbon composite may further include crystalline carbon. For example, the silicon-carbon composite may include a core including crystalline carbon and silicon particles and may also include an amorphous carbon coating layer on (e.g., positioned on) a surface of the core.

In one or more embodiments, the Si-based negative electrode active material and/or the Sn-based negative electrode active material may be used in combination with a carbon-based negative electrode active material.

### Separator 30

Based on type (kind) of the rechargeable lithium battery, the separator 30 may be present between the positive electrode 10 and the negative electrode 20. The separator 30 may include one or more selected from among polyethylene, polypropylene, and polyvinylidene fluoride, or may have a multi-layered separator thereof such as a polyethylene/polypropylene bi-layered separator, a polyethylene/polypropylene/polyethylene tri-layered separator, or a polypropylene/polyethylene/polypropylene tri-layered separator.

The separator 30 may include a porous substrate and a coating layer on (e.g., positioned on) a surface (e.g., one surface or two opposite surfaces) of the porous substrate, and the coating layer may include an organic material, an inorganic material, and/or a (e.g., any suitable) combination thereof.

The porous substrate may be a polymer layer including one selected from among polyolefin such as polyethylene and polypropylene, polyester such as polyethylene terephthalate and polybutylene terephthalate, polyacetal, polyamide, polyimide, polycarbonate, polyetherketone, polyaryletherketone, polyetherimide, polyamideimide, polybenzimidazole, polyethersulfone, polyphenyleneoxide, cyclic olefin copolymers, polyphenylenesulphide, polyethylene naphthalate, glass fibers, and polytetrafluoroethylene (e.g., Teflon), or may be a copolymer or a mixture including two or more thereof.

The organic material may include a polyvinylidenefluoride-based copolymer and/or a (meth)acrylic copolymer.

The inorganic material may include an inorganic particle selected from among Al₂O₃, SiO₂, TiO₂, SnO₂, CeO₂, MgO, NiO, CaO, GaO, ZnO, ZrO₂, Y₂O₃, SrTiO₃, BaTiO₃, Mg(OH)₂, Boehmite, and/or a (e.g., any suitable) combination thereof, but embodiments of the present disclosure are not limited thereto.

In one or more embodiments, the organic material and the inorganic material may be present mixed in one coating layer or may be present as a stack of a coating layer including the organic material and a coating layer including an inorganic material.

### Electrolyte ELL

The electrolyte ELL for the rechargeable lithium battery may include a non-aqueous organic solvent and a lithium salt.

The non-aqueous organic solvent may serve as a medium for transmitting ions that participate in an electrochemical reaction of the battery.

The non-aqueous organic solvent may include a carbonate-based solvent, an ester-based solvent, an ether-based solvent, a ketone-based solvent, an alcohol-based solvent, an aprotic solvent, and/or a (e.g., any suitable) combination thereof.

The carbonate-based solvent may include dimethyl carbonate (DMC), diethyl carbonate (DEC), dipropyl carbonate (DPC), methylpropyl carbonate (MPC), ethylpropyl carbonate (EPC), methylethyl carbonate (MEC), ethylene carbonate (EC), propylene carbonate (PC), and/or butylene carbonate (BC).

The ester-based solvent may include methyl acetate, ethyl acetate, n-propyl acetate, dimethyl acetate, methyl propionate, ethyl propionate, decanolide, mevalonolactone, valerolactone, caprolactone, and/or propyl propionate (PP).

The ether-based solvent may include dibutyl ether, tetraglyme, diglyme, dimethoxyethane, 2-methyltetrahydrofuran, 2.5-dimethyltetrahydrofuran, and/or tetrahydrofuran. The ketone-based solvent may include cyclohexanone. The alcohol-based solvent may include ethyl alcohol and/or isopropyl alcohol. The aprotic solvent may include nitriles such as R-CN (where R is a hydrocarbon group having a C2 to C20 linear, branched, or cyclic structure and may include a double bond, an aromatic ring, or an ether group); amides such as dimethylformamide; dioxolanes such as 1,3-dioxolane and/or 1.4-dioxolane; and/or sulfolanes.

The non-aqueous organic solvent may be used alone or in a mixture of two or more thereof.

In addition, if (e.g., when) a carbonate-based solvent is used, a cyclic

carbonate and a chain carbonate may be mixed and used, and the cyclic carbonate and the chain carbonate may be mixed in a volume ratio of about 1:1 to about 1:9.

The lithium salt may be a material that is dissolved in the non-aqueous organic solvent to serve as a supply source of lithium ions in a rechargeable lithium battery and plays a role in enabling a basic operation of the rechargeable lithium battery and in promoting the movement of lithium ions between the positive electrode and the negative electrode. The lithium salt may include, for example, at least one selected from among LiPF₆, LiBF₄, LiSbF₆, LiAsF₆, LiClO₄, LiAlO₂, LiAlCl₄, LiPO₂F₂, LiCl, LiI, LiN (SO₃C₂F₅)₂, Li(FSO₂)₂N (lithium bis(fluorosulfonyl)imide, LiFSl), LiC₄F₉SO₃, LiN(CₓF₂ₓ₊₁SO₂)(C_{y}F_{2y+1}SO₂) (where x and y are integers of 1 to 20), lithium trifluoromethane sulfonate, lithium tetrafluoroethanesulfonate, lithium difluoro(oxalato)borate (LiDFOB), lithium difluorobis (oxalato) phosphate (LiDFBOP), and lithium bis(oxalato) borate (LiBOB).

Hereinafter, the electrolyte of a rechargeable lithium battery according to one or more embodiments of the present disclosure will be explained in more detail.

The electrolyte for a rechargeable lithium battery according to one or more embodiments may include a non-aqueous organic solvent, a lithium salt, and an additive, and the additive may be represented by Formula 1.

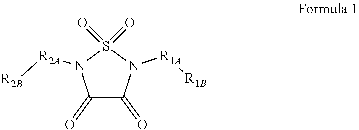

In Formula 1,

R_{1A} and R_{2A} may each independently be a substituted or unsubstituted C1 to C10 alkylene group, a substituted or unsubstituted C2 to C10 alkenylene group, or a substituted or unsubstituted C2 to C10 alkynylene group,

R_{1B} and R_{2B} may each independently be hydrogen, a substituted or unsubstituted C1 to C10 alkyl group, a substituted or unsubstituted C2 to C10 alkenyl group, or a substituted or unsubstituted C2 to C10 alkynyl group, and at least one selected from among R_{1B} and R_{2B} may include an alkenyl group.

Detailed explanation on the additive will be provided separately later.

The electrolyte may be prepared by a method including dissolving a lithium salt in a non-aqueous organic solvent, adding the additive, and performing a mixing process. The mixing process of the electrolyte may be widely available in the field of preparing an electrolyte, and a person skilled in the art may select and use it appropriately or suitably. In other words, a person skilled in the art should be able to select and apply the appropriate or suitable mixing method as needed.

In one or more embodiments, the non-aqueous organic solvent may include ethylene carbonate (EC), ethyl methyl carbonate (EMC), and dimethyl carbonate (DMC).

The ethylene carbonate (EC) may be included in about 20 vol % to about 30vol % on the basis of 100 vol % of a total volume of the non-aqueous organic solvent. The ethyl methyl carbonate (EMC) may be included in about 0.01 vol % to about 52vol % on the basis of 100 vol % of the total volume of the non-aqueous organic solvent. The dimethyl carbonate (DMC) may be included in about 60 vol % to about 80 vol % on the basis of 100 vol % of the total volume of the non-aqueous organic solvent. In one or more embodiments, the ethylene carbonate (EC) may be included in about 15 vol % to about 25 vol % on the basis of 100 vol % of the total volume of the non-aqueous organic solvent. The ethyl methyl carbonate (EMC) may be included in about 5 vol % to about 15 vol % on the basis of 100 vol % of the total volume of the non-aqueous organic solvent. The dimethyl carbonate (DMC) may be included in about 65 vol % to about 75 vol % on the basis of 100 vol % of the total volume of the non-aqueous organic solvent.

The ethylene carbonate (EC), ethyl methyl carbonate (EMC), and dimethyl carbonate (DMC) may have a volume ratio of about 1:a:b (i.e., the volume ratio of EC:EMC:DMC is about 1:a:b). “a” may be 0.01 to 1.5, and “b” may be 2.5 to 4.5. If (e.g., when) the types (kinds) and the volume ratio of the organic solvents are satisfied, the solubility of the additive may be appropriately or suitably maintained. However, example embodiments are mere illustrations of the present disclosure and embodiments of the present disclosure are not limited thereto.

In one or more embodiments, the lithium salt may be one or two or more selected from among LiPF₆, LiBF₄, LiSbF₆, LiAsF₆, LiClO₄, LiAlO₂, LiAlCl₄, LiPO₂F₂, LiCl, LiI, LiN(SO₃C₂F₅)₂, lithium bis(fluorosulfonyl)imide (Li(FSO₂)₂N, LiFSl)), LiC₄F₉SO₃, LiN(CₓF₂ₓ₊₁SO₂) (C_{y}F_{2y+1}SO₂) (x and y are integers of 1 to 20), lithium trifluoromethanesulfonate, lithium tetrafluoroethanesulfonate, lithium difluoro(oxalato)borate (LiDFOB), lithium difluorobis(oxalato)phosphate (LiDFBOP) and lithium bis(oxalato)borate (LiBOB).

A concentration of the lithium salt may be about 0.1 M to about 2.0 M. For example, in one or more embodiments, the concentration of the lithium salt may be about 0.5 M or more, or about 1.0 M or more. The concentration of the lithium salt may be about 2.0 M or less, about 1.7 M or less, or about 1.5 M or less. If the concentration of the lithium salt is in a range of about 0.1 M to about 2.0 M, the conductivity of the electrolyte and the viscosity of the electrolyte may be appropriately or suitably maintained.

### Additive

The additive according to one or more embodiments of the present disclosure may include a compound represented by Formula 1.

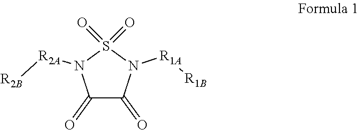

In Formula 1,

R_{1A} and R_{2A} may each independently be a substituted or unsubstituted C1 to C10 alkylene group, a substituted or unsubstituted C2 to C10 alkenylene group, or a substituted or unsubstituted C2 to C10 alkynylene group,

R_{1B} and R_{2B} may each independently be hydrogen, a substituted or unsubstituted C1 to C10 alkyl group, a substituted or unsubstituted C2 to C10 alkenyl group, or a substituted or unsubstituted C2 to C10 alkynyl group, and at least one selected from among R1B and R2B may include an alkenyl group.

In one or more embodiments, R_{1A} and R_{2A} may each independently be a substituted or unsubstituted C1 to C5 alkylene group, a substituted or unsubstituted C2to C5 alkenylene group, or a substituted or unsubstituted C2 to C5 alkynylene group. In one or more embodiments, R_{1A} and R2A may each independently be a substituted or unsubstituted C1 to C5 alkylene group.

In one or more embodiments, R_{1B} and R_{2B} may each independently be hydrogen, a substituted or unsubstituted C1 to C5 alkyl group, a substituted or unsubstituted C2 to C5 alkenyl group, or a substituted or unsubstituted C2 to C5alkynyl group. In one or more embodiments, R_{1B} and R_{2B} may each independently be a substituted or unsubstituted C2 to C5 alkenyl group.

In one or more embodiments, R_{1B} and R_{2B} may each include an alkenyl group.

In one or more embodiments, Formula 1 may be Formula 2-1 or Formula 2-2. For example, the additive according to one or more embodiments of the present disclosure may include a compound of Formula 2-1, a compound of Formula 2-2, and/or a (e.g., any suitable) combination thereof.

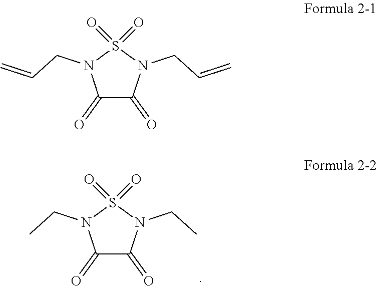

A content (e.g., an amount) of the additive may be in a range of about 0.01 vol % to about 10 vol % on the basis of 100 vol % of a total volume of the electrolyte for a rechargeable lithium battery. In one or more embodiments, the content (e.g., amount) of the additive may be in a range of about 0.01 vol % to about 5 vol %, or about 0.25 vol % to about 1 vol %, on the basis of 100 vol % of the total volume of the electrolyte for a rechargeable lithium battery. The content (e.g., amount) of the additive may refer to the volume of the additive contained in the electrolyte with respect to the total volume of the electrolyte. If (e.g., when) the content (e.g., amount) of the additive satisfies the above-described range, the improving effects on the performance of a rechargeable lithium battery at high temperatures may be maximized or increased.

The compound represented by Formula 1 may have a structure including a “oxalic acid derivative moiety”, a “terminal alkenyl moiety,” and a “sulfonyl moiety.”

The “oxalic acid derivative moiety” may refer to a moiety including a structure that constitutes “nitrogen (N)-carbonyl group (C═O)-carbonyl group (C=O)-nitrogen (N).” The “oxalic acid derivative moiety” may be characterized in introducing nitrogen (N) instead of oxygen (O) into oxalic acid.

Lithium bis (oxalato) borate (LiBOB), which is widely used as an additive to

improve battery performance, has a chronic defect of gas generation. The additive according to the present disclosure may have the effect of significantly suppressing or reducing gas generation by replacing oxygen (O), which causes the gas generation, with nitrogen (N).

The “sulfonyl moiety” may refer to a moiety including “—SO—.” The sulfonyl

group may be reduced and decomposed on the surface of a negative electrode to form a solid electrolyte interface (SEI) that is strong and has excellent or suitable ion conductivity on the surface of the negative electrode. A stable SEI may suppress or reduce the decomposition of the surface of the negative electrode and thus suppress or reduce the increase in battery resistance.

The “terminal alkenyl moiety” may refer to an alkenyl moiety positioned at the terminal of the compound of Formula 1. The terminal of the compound may refer to R_{1B} and R_{2B}. The alkenyl group may include, for example, a vinyl group, a propenyl group, an isopropenyl group, and/or the like. In one or more embodiments, the “terminal alkenyl moiety” may be a vinyl group. The compound of Formula 1 may include at least one or more “terminal alkenyl moieties.” In one or more embodiments, two “terminal alkenyl moieties” may be included. The compound of Formula 1 may participate in the formation of a SEI on the surface of the negative electrode and improve the stability of the SEI. Because the alkenyl moiety is positioned at the terminal of the structure of the compound, the reaction that stabilizes the SEI may occur more easily.

Because the compound represented by Formula 1 has the structural characteristics described above, the additive according to the present disclosure may have excellent or suitable effects on improving the performance of a rechargeable lithium battery if (e.g., when) activating (e.g., charging) the rechargeable lithium battery. The effect may be more excellent or suitable at high temperatures and/or during rapid charging. The high temperature may refer to about 40° C. or higher, about 50° C. or higher, or about 60° C. or higher.

In other words, the additive described in this disclosure includes a compound represented by Formula 1. This compound features specific groups, such as nitrogen-containing oxalic acid derivative moiety, terminal alkenyl moiety, and sulfonyl moiety. The additive's composition and structure are designed to enhance the performance of rechargeable lithium batteries, particularly at high temperatures and during rapid charging, by improving the stability and conductivity of the solid electrolyte interface (SEI) on the negative electrode.

### Rechargeable Lithium Battery

A rechargeable lithium battery may be classified into a cylindrical, prismatic,

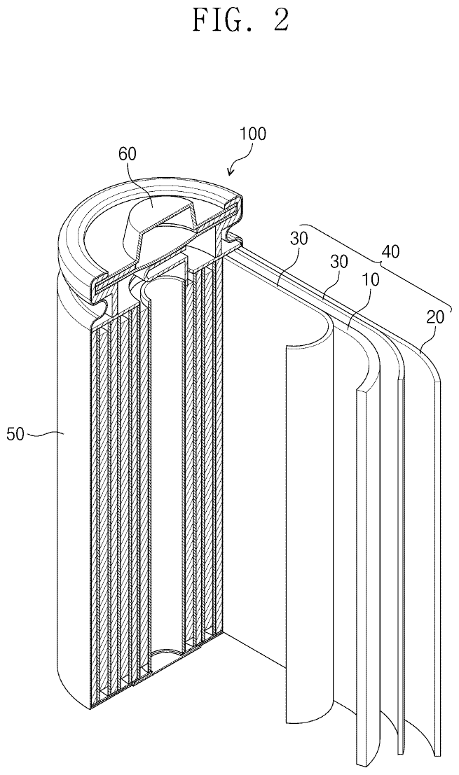

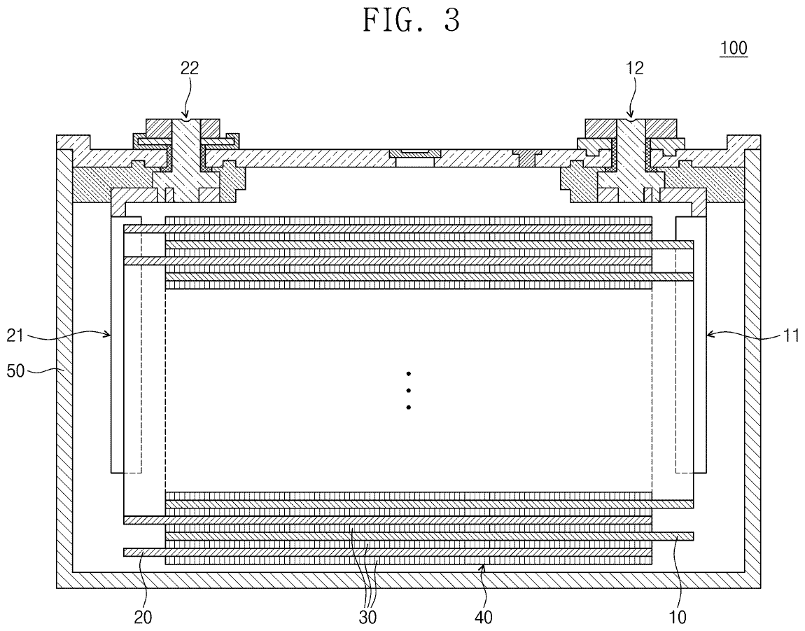

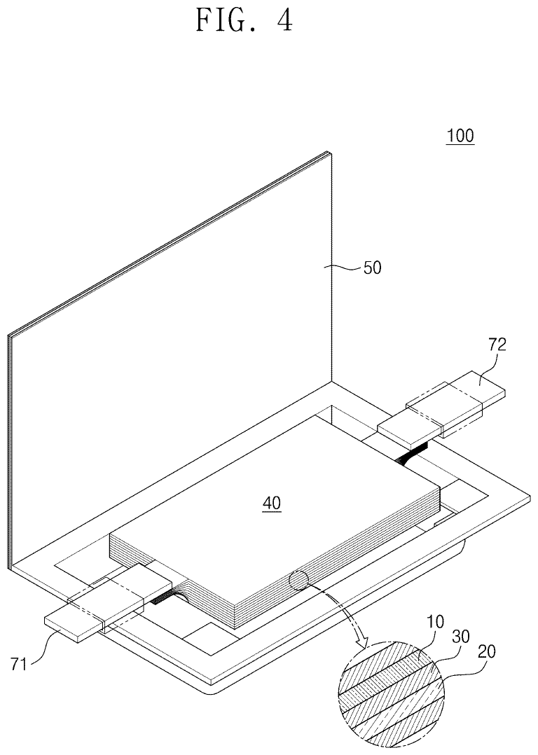

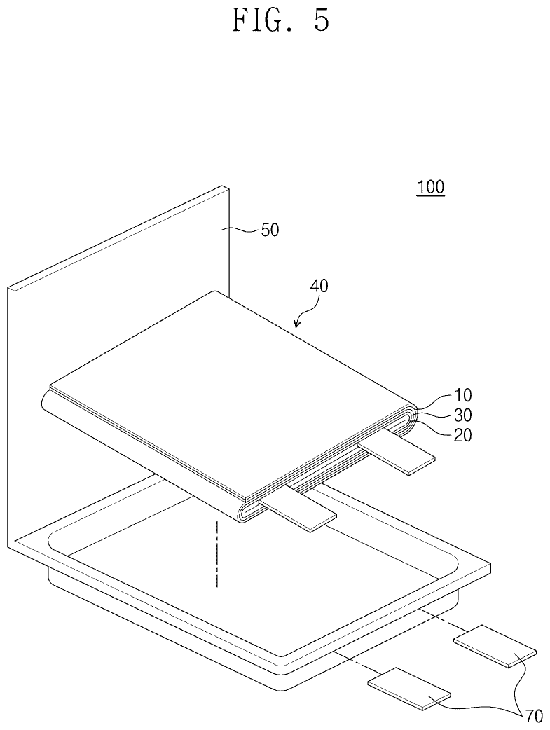

pouch, or coin-type (kind) battery depending on the shape thereof (e.g., external form thereof). FIGS. 2 to 5 are each a schematic view illustrating a rechargeable lithium battery according to one or more embodiments of the present disclosure, where FIG. 2 shows a cylindrical battery, FIG. 3 shows a prismatic battery, and FIGS. 4 and 5 each show a pouch-type (kind) battery. Referring to FIGS. 2 to 5, a rechargeable lithium battery (100) may include an electrode assembly (40) including a separator (30) arranged between a positive electrode (10) and a negative electrode (20), and a case (50) accommodating the electrode assembly (40). The positive electrode (10), the negative electrode (20), and the separator (30) may be immersed in an electrolyte. In one or more embodiments, the rechargeable lithium battery (100) may include a sealing member (60) sealing the case (50), as shown in FIG. 2. In one or more embodiments, as shown in FIG. 3, the rechargeable lithium battery (100) may include a positive electrode lead tab (11), a positive terminal (12), a negative electrode lead tab (21), and a negative terminal (22). In one or more embodiments, as shown in FIGS. 4 and 5, the rechargeable lithium battery (100) may include an electrode tab (70), or a positive electrode tab (71) and a negative electrode tab (72), serving as an electrical path for inducing the current formed in the electrode assembly (40) to the outside.

The rechargeable lithium battery according to one or more embodiments of

the present disclosure may be applied to automobiles, mobile phones, and/or one or more suitable types (kinds) of electric devices, but embodiments of the present disclosure are not limited thereto.

The rechargeable lithium battery according to one or more embodiments of the present disclosure may include a positive electrode including a positive electrode active material, a negative electrode including a negative electrode active material, and the electrolyte for a rechargeable lithium battery.

The positive electrode active material may include a lithium composite oxide represented by Formula 3.

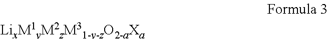

0.5≤x≤1.8, 0≤a≤0.05, 0<y≤1, 0≤z≤1, and 1≤y+z≤1,

M¹, M², and M³ may each independently include one or more elements selected from among metals including Ni, Co, Mn, Al, B, Ba, Ca, Ce, Cr, Fe, Mo, Nb, Si, Sr, Mg, Ti, V, W, Zr, and La, and/or a (e.g., any suitable) combination thereof, and X may include one or more elements selected from among F, S, P and Cl.

In one or more embodiments, in Formula 3, M1 may be Ni. In one or more embodiments, in Formula 3, M¹ may be Ni, M² may be Co, and M³ may be Al. For example, the positive electrode active material may include a nickel-cobalt-aluminumNCA)-based positive electrode active material.

A positive electrode active material including Ni may induce significant side reactions with an electrolyte due to the inherent instability of the Ni element. Accordingly, the battery stabilization effect of the additive according to the present disclosure may be more significantly exhibited in the positive electrode active material. In other words, the additive according to the present disclosure may significantly stabilizes a positive electrode active material including Ni.

The negative electrode active material may be a carbon-based negative electrode active material, a Si-based negative electrode active material, a Sn-based negative electrode active material, or a (e.g., any suitable) combination thereof.

Hereinafter, examples and comparative examples of the present disclosure will be described. However, these examples are mere example embodiments of the present disclosure, and the present disclosure is not limited to these examples.

### Synthetic Example 1

To a round-bottom flask, 20.0 g of allylamine was added, and 100 g of dichloromethane was added thereto, and then, the temperature was controlled or selected to be about −5° C. while stirring. 35.5 g of triethylamine was added thereto dropwise and stirred for about 30 minutes. A solution in which 23.6 g of sulfuryl chloride was diluted in 100 g of dichloromethane was slowly added thereto dropwise, and the temperature was raised to room temperature (about 25° C.), followed by stirring for about 1 hour. After finishing the reaction, a salt was removed by filtration, washed with water and precipitated with hexane to obtain a compound of Formula A.

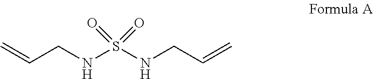

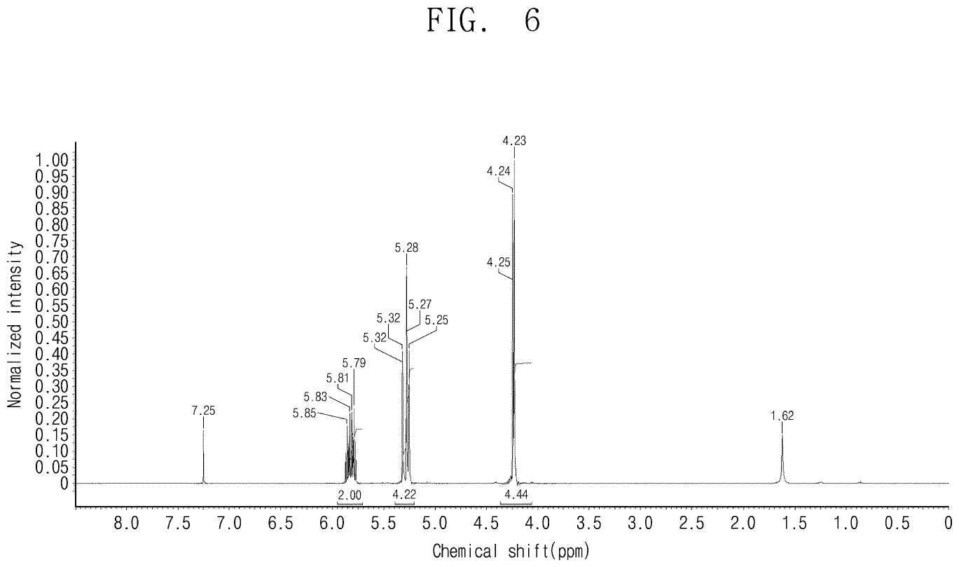

10.0 g of the compound of Formula A thus obtained was added to a round-bottom flask, and 70 g of dichloromethane was added thereto, followed by stirring at room temperature. 10.8 g of oxalyl chloride was dissolved in 30 g of dichloromethane, and the resultant was slowly added to the round-bottom flask dropwise. The temperature was raised to about 40° C., and stirring was performed for about 2 hours. After finishing the reaction, the temperature was lowered to room temperature, and the reaction product was washed with water, dried and filtered to obtain a compound represented by Formula 2-1. The compound of Formula 2-1 was confirmed by the proton nuclear magnetic resonance (¹H-NMR) spectroscopy as shown in FIG. 6.

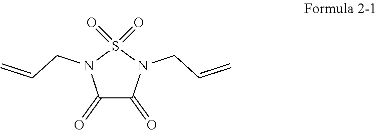

### Synthetic Example 2

A compound represented by Formula 2-2 was obtained by performing substantially the same method as in Synthetic Example 1 except for using ethylamine instead of allylamine.

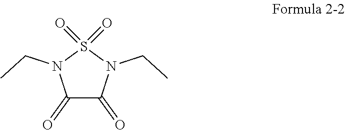

### EXAMPLE 1

(1) Preparation of Electrolyte

In a non-aqueous organic solvent in which ethylene carbonate (EC), ethyl methyl carbonate (EMC), and dimethyl carbonate (DMC) were mixed in a volume ratio of about 20:10:70, about 1.25 M of LiPF₆ was dissolved. An additive including the compound represented by Formula 2-1 prepared in Synthetic Example 1 was added thereto to prepare an electrolyte. The content (e.g., amount) of the additive was about 0.25 vol % on the basis of 100 vol % of the total volume of the electrolyte for a rechargeable lithium battery.
(2) Fabrication of Rechargeable Lithium Battery

LiNi_{0.91}Co_{0.07}Al_{0.02}O₂ as a positive electrode active material, polyvinylidene fluoride as a binder, and Ketjen black as a conductive material were mixed in a weight ratio of about 97:2:1 and dispersed in N-methyl pyrrolidone to prepare a positive electrode active material slurry.

The positive electrode active material slurry was applied on an aluminum current collector with a thickness of about 14 micrometers (μm), dried at about 110° C. and pressed to manufacture a positive electrode.

A mixture of artificial graphite and silicon nano particles in a weight ratio of about 93:7 as a negative electrode active material, styrene-butadiene rubber (SBR) as a binder, and carboxymethyl cellulose (CMC) as a thickening agent were mixed in a weight ratio of about 97:1:2, and dispersed in distilled water to prepare a negative electrode active material slurry.

The negative electrode active material slurry was applied on a copper current collector with a thickness of about 10 μm, dried at about 100° C. and pressed to manufacture a negative electrode.

The positive electrode, the negative electrode, and a separator of a polyethylene material with a thickness of about 25 μm were assembled to fabricate an electrode assembly, and an electrolyte was injected to fabricate a rechargeable lithium battery.

### EXAMPLE 2

An electrolyte and a rechargeable lithium battery were obtained by substantially the same method as in Example 1 except that the content (e.g., amount) of the compound represented by Formula 2-1 was about 0.5 vol %.

### EXAMPLE 3

An electrolyte and a rechargeable lithium battery were obtained by substantially the same method as in Example 1 except that the content (e.g., amount) of the compound represented by Formula 2-1 was about 1.0 vol %.

### Example 4

An electrolyte and a rechargeable lithium battery were obtained by substantially the same method as in Example 1 except that the content (e.g., amount) of the compound represented by Formula 2-1 was about 5.0 vol %.

### EXAMPLE 5

An electrolyte and a rechargeable lithium battery were obtained by substantially the same method as in Example 1 except that the compound represented by Formula 2-2 was used instead of the compound represented by Formula 2-1.

### COMPARATIVE EXAMPLE 1

An electrolyte and a rechargeable lithium battery were obtained by substantially the same method as in Example 1 except that the electrolyte did not include the compound represented by Formula 2-1.

### Evaluation Example 1: Evaluation of Resistance (DC-IR) Increase ratio during High Temperature Storage

Each of the rechargeable lithium batteries fabricated in the Examples and Comparative Example was charged under conditions of about 25° C., about 1.0 C-rate, and about 4.25 V and about 0.05 C-rate cut-off, and the initial resistance (DC-IR) value of each of the batteries was measured. The resistance value of each of the batteries after being left at about 60° C. for 30 days was measured. The resistance was measured using electrochemical impedance spectroscopy (EIS). The resistance increase ratios were calculated according to Equation 1, and the results are shown in Table 1.

$$Resistance increase ratio ( \% ) = [ DC - IR of battery after 30 days / DC - IR of initial battery ] ⋆ 100 \qquad Equation 1$$

### Evaluation Example 2: Evaluation of Gas Generation Amount during High Temperature Storage

Each of the rechargeable lithium batteries of the Examples and Comparative Example was charged under conditions of about 25° C., about 1.0 C-rate, and about 4.25 V and about 0.05 C-rate cut-off, and initial gas generation amount of each of the batteries was measured. The batteries were each left at about 60° C. for 7 days, and the gas generation amount of each of the batteries was measured. The results are shown in Table 1.

### Evaluation Example 3: Evaluation of High Temperature Capacity Retention

With respect to each of the rechargeable lithium batteries of the Examples and Comparative Example, capacity retention was calculated by performing 200 cycles of charge and discharge at high temperatures. Charge conditions were about 60° C., about 1.0 C-rate, and about 4.25 V and about 0.05 C-rate cut-off. Discharge conditions were about 60° C., about 1.0 C-rate, and about 2.8 V cut-off. The capacity retention was calculated according to Equation 2. The results are shown in Table 1.

$$Capacity retention ( \% ) = ( disharge capacity after 200 cycles / disharge capacity after 1 cycle ) ⋆ 100 \qquad Equation 2$$

TABLE 1

| | Additive | Evaluation results | | | | | |
|---|---|---|---|---|---|---|---|
| | Formula | | | | Gas | Gas | |
| | [content | | DC-IR | DC-IR | generation | generation | |
| | (e.g., | Initial | after | increase | amount | amount | Capacity |
| | amount)] | DC-IR | 30 days | ratio | (Day 1) | (Day 7) | retention |
| | (vol %) | (mOhm) | (mOhm) | (%) | (mL) | (mL) | (%) |
| Comparative | — [—] | 8.28 | 10.15 | 123 | 0.033 | 0.080 | 89.1 |
| Example 1 | | | | | | | |
| Example 1 | Formula | 8.34 | 8.74 | 105 | 0.031 | 0.052 | 95.4 |
| | 2-1 [0.25] | | | | | | |
| Example 2 | Formula | 8.74 | 9.14 | 105 | 0.028 | 0.049 | 95.3 |
| | 2-1 [0.5] | | | | | | |
| Example 3 | Formula | 9.24 | 10.15 | 110 | 0.016 | 0.042 | 94.8 |
| | 2-1 [1.0] | | | | | | |
| Example 4 | Formula | 10.04 | 10.28 | 102 | 0.008 | 0.017 | 93.5 |
| | 2-1 [5.0] | | | | | | |
| Example 5 | Formula | 8.30 | 8.80 | 106 | 0.038 | 0.059 | 95.2 |
| | 2-2 [0.25] | | | | | | |

Referring to Table 1, it was confirmed that each of the Examples according to the present disclosure showed excellent or suitable evaluation results on the resistance increase ratio, gas generation amount, and capacity retention, compared to the Comparative Example. For example, it was confirmed that the additive according to the present disclosure has excellent or suitable improving effects on battery performance at high temperatures.

The additive according to one or more embodiments of disclosure may have excellent or suitable improving effects on battery performance at high temperatures.

A rechargeable lithium battery including the additive may have excellent or suitable performance at high temperatures.

In the present disclosure, expressions such as “at least one of,” “one of,” and “selected from,” when preceding a list of elements, modify the entire list of elements and do not modify the individual elements of the list. For example, “at least one of a, b or c”, “at least one selected from a, b, and c”, “at least one selected from among a to c”, etc., may indicate only a, only b, only c, both (e.g., simultaneously) a and b, both (e.g., simultaneously) a and c, both (e.g., simultaneously) b and c, all of a, b, and c, or variations thereof. The “/” utilized herein may be interpreted as “and” or as “or” depending on the situation.

In the context of the present disclosure and unless otherwise defined, the terms “use,” “using,” and “used” may be considered synonymous with the terms “utilize,” “utilizing,” and “utilized,” respectively.

In present disclosure, The term “Group” as utilized herein refers to a group of

the Periodic Table of Elements according to the 1 to 18 grouping system of the International Union of Pure and Applied Chemistry (“IUPAC”).

As used herein, the terms “substantially,” “about,” and similar terms are used as terms of approximation and not as terms of degree, and are intended to account for the inherent deviations in measured or calculated values that would be recognized by those of ordinary skill in the art. “About” or “approximately,” as used herein, is also inclusive of the stated value and means within an acceptable range of deviation for the particular value as determined by one of ordinary skill in the art, considering the measurement in question and the error associated with measurement of the particular quantity (i.e., the limitations of the measurement system). For example, “about” may mean within one or more standard deviations, or within ±30%, 20%, 10%, or 5% of the stated value.

Any numerical range recited herein is intended to include all sub-ranges of the same numerical precision subsumed within the recited range. For example, a range of “1.0 to 10.0” is intended to include all subranges between (and including) the recited minimum value of 1.0 and the recited maximum value of 10.0, that is, having a minimum value equal to or greater than 1.0 and a maximum value equal to or less than 10.0, such as, for example, 2.4 to 7.6. Any maximum numerical limitation recited herein is intended to include all lower numerical limitations subsumed therein and any minimum numerical limitation recited in this specification is intended to include all higher numerical limitations subsumed therein. Accordingly, Applicant reserves the right to amend this specification, including the claims, to expressly recite any sub-range subsumed within the ranges expressly recited herein.

A battery manufacturing device, a battery management system (BMS) device, and/or any other relevant devices or components according to embodiments of the present invention described herein may be implemented utilizing any suitable hardware, firmware (e.g., an application-specific integrated circuit), software, or a combination of software, firmware, and hardware. For example, the various components of the device may be formed on one integrated circuit (IC) chip or on separate IC chips. Further, the various components of the device may be implemented on a flexible printed circuit film, a tape carrier package (TCP), a printed circuit board (PCB), or formed on one substrate. Further, the various components of the device may be a process or thread, running on one or more processors, in one or more computing devices, executing computer program instructions and interacting with other system components for performing the various functionalities described herein. The computer program instructions are stored in a memory which may be implemented in a computing device using a standard memory device, such as, for example, a random access memory (RAM). The computer program instructions may also be stored in other non-transitory computer readable media such as, for example, a CD-ROM, flash drive, or the like. Also, a person of skill in the art should recognize that the functionality of various computing devices may be combined or integrated into a single computing device, or the functionality of a particular computing device may be distributed across one or more other computing devices without departing from the scope of the present disclosure.

A person of ordinary skill in the art would appreciate, in view of the present disclosure in its entirety, that each suitable feature of the various embodiments of the present disclosure may be combined or combined with each other, partially or entirely, and may be technically interlocked and operated in various suitable ways, and each embodiment may be implemented independently of each other or in conjunction with each other in any suitable manner unless otherwise stated or implied.

Although one or more embodiments of the present disclosure have been described, it is understood that the present disclosure should not be limited to these embodiments but one or more suitable changes and modifications can be made by one ordinary skilled in the art within the spirit and scope of the disclosure as hereinafter claimed and equivalents thereof.

1 what is claimed is:

1. An electrolyte, comprising:
a non-aqueous organic solvent;
a lithium salt; and
an additive,
wherein the additive comprises a compound represented by Formula 1: 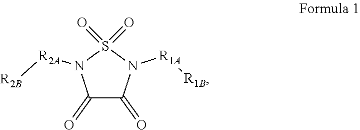
in Formula 1,
R_{1A} and R_{2A} are each independently a substituted or unsubstituted C1 to C10alkylene group, a substituted or unsubstituted C2 to C10 alkenylene group, or a substituted or unsubstituted C2 to C10 alkynylene group,
R_{1B} and R_{2B} are each independently hydrogen, a substituted or unsubstituted C1 to C10 alkyl group, a substituted or unsubstituted C2 to C10 alkenyl group, or a substituted or unsubstituted C2 to C10 alkynyl group, and at least one selected from among R_{1B} and R_{2B} comprises an alkenyl group, and wherein the electrolyte is for a rechargeable lithium battery.

2. The electrolyte as claimed in claim 1, wherein R_{1A} and R_{2A} are each independently a substituted or unsubstituted C1 to C5 alkylene group, a substituted or unsubstituted C2 to C5 alkenylene group, or a substituted or unsubstituted C2 to C5alkynylene group.

3. The electrolyte as claimed in claim 2, wherein R_{1A} and R_{2A} are each independently a substituted or unsubstituted C1 to C5 alkylene group.

4. The electrolyte as claimed in claim 1, wherein R_{1B} and R_{2B} are each independently hydrogen, a substituted or unsubstituted C1 to C5 alkyl group, a substituted or unsubstituted C2 to C5 alkenyl group, or a substituted or unsubstituted C2 to C5 alkynyl group.

5. The electrolyte as claimed in claim 4, wherein R_{1B} and R_{2B} are each independently a substituted or unsubstituted C2 to C5 alkenyl group.

6. The electrolyte as claimed in claim 1, wherein R_{1B} and R₂₈ each comprise an alkenyl group.

7. The electrolyte as claimed in claim 1, wherein an amount of the additive is in a range of about 0.01 vol % to about 10 vol % on the basis of 100 vol % of a total volume of the electrolyte.

8. The electrolyte as claimed in claim 1, wherein an amount of the additive is in a range of about 0.01 vol % to about 5 vol % on the basis of 100 vol % of a total volume of the electrolyte.

9. The electrolyte as claimed in claim 1, wherein the non-aqueous organic solvent comprises ethylene carbonate, ethyl methyl carbonate, and dimethyl carbonate.

10. The electrolyte as claimed in claim 9, wherein the ethylene carbonate, the ethyl methyl carbonate, and the dimethyl carbonate have a volume ratio of about 1:a:b, where
“a” is 0.01 to 1.5, and
“b” is 2.5 to 4.5.

11. The electrolyte as claimed in claim 1, wherein the lithium salt is one or more selected from among LiPF₆, LiBF₄, LiSbF₆, LiAsF₆, LiClO₄, LiAlO₂, LiAlCl₄, LiPO₂F₂, LiCl, LiI, LiN (SO₃C₂F₅)₂, lithium bis (fluorosulfonyl)imide (Li(FSO₂)₂N), LiC₄F₉SO₃, LiN(CₓF₂ₓ₊₁SO₂)(CyF_{2y+1}SO₂)(x and y are integers of 1 to 20), lithium trifluoromethanesulfonate, lithium tetrafluoroethanesulfonate, lithium difluoro (oxalato) borate, lithium difluorobis (oxalato) phosphate. and lithium bis (oxalato) borate.

12. The electrolyte as claimed in claim 1, wherein the lithium salt comprises LiPF6.

13. The electrolyte as claimed in claim 1, wherein a concentration of the lithium salt is in a range of about 0.1 M to about 2.0 M.

14. A compound represented by Formula 2-1 or Formula 2-2:

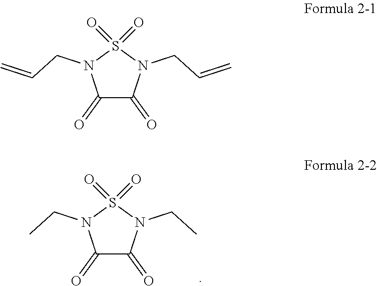

15. A rechargeable lithium battery, comprising:
a positive electrode comprising a positive electrode active material;
a negative electrode comprising a negative electrode active material; and
the electrolyte as claimed in claim 1.

16. The rechargeable lithium battery as claimed in claim 15, wherein the positive electrode active material comprises a lithium composite oxide represented by Formula 3: 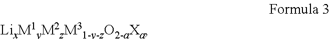
where 0.5≤x≤1.8, 0≤a≤0.05, 0<y≤1, 0z≤1, and 1≤y+z≤1,
M¹, M², and M³ each independently comprises one or more elements selected from among metals comprising Ni, Co, Mn, Al, B, Ba, Ca, Ce, Cr, Fe, Mo, Nb, Si, Sr, Mg, Ti, V, W, Zr, and La, and a combination thereof, and
X comprises one or more elements selected from among F, S, P, and Cl.

17. The rechargeable lithium battery as claimed in claim 16, wherein, in Formula 3, M¹ is Ni.

18. The rechargeable lithium battery as claimed in claim 15, wherein the positive electrode active material comprises a nickel-cobalt-aluminum-based positive electrode active material.

19. The rechargeable lithium battery as claimed in claim 15, wherein the negative electrode active material comprises a carbon-based negative electrode active material, a Si-based negative electrode active material, a Sn-based negative electrode active material, or a combination thereof.

20. The rechargeable lithium battery as claimed in claim 15, wherein an external form of the rechargeable lithium battery has a cylindrical shape or a prismatic shape.
